(19) United States
(12) Patent Application Publication
(10) Pub. No.: US 2022/0234973 A1
(43) Pub. Date: Jul. 28, 2022

(54) METHOD AND FACILITY FOR PRODUCING A TARGET COMPOUND

(71) Applicant: LINDE GMBH, Pullach (DE)

(72) Inventors: Andreas MEISWINKEL, Rimsting (DE); Hans-Jörg ZANDER, München (DE); Ernst HAIDEGGER, Riemerling (DE)

(21) Appl. No.: 17/627,802

(22) PCT Filed: Jul. 16, 2020
(86) PCT No.: PCT/EP2020/070191

§ 371 Date: Jan. 17, 2022

(30) Foreign Application Priority Data
Jul. 18, 2019 (DE) 10 2019 119 540.3

(51) Int. Cl. C07C 29/141; C01B 3/34; C01B 3/48; C07C 45/50; C07C 1/24

(52) U.S. Cl. CPC C07C 29/141; C01B 3/34; C01B 3/48; C01B 2203/1241; C07C 1/24; C01B 2203/0238; C01B 2203/063; C07C 45/50

(57) ABSTRACT

The invention relates to a method (100) for producing a target compound, wherein a paraffin is subjected to an oxidative dehydrogenation (1) with oxygen to obtain an olefin, and wherein the olefin is subjected to a hydroformylation (2) with carbon monoxide to obtain an aldehyde, wherein the paraffin and the olefin have a carbon chain having a first carbon number and the aldehyde has a carbon chain having a second carbon number which is one greater than the first carbon number. It is provided that carbon dioxide is formed as a by-product in the oxidative dehydrogenation (1), that the carbon dioxide is subjected to dry reforming (3) at least in part with methane to obtain carbon monoxide and hydrogen, and that the carbon monoxide obtained in the dry reforming (3) and/or the hydrogen obtained in the dry reforming (3) is supplied to the hydroformylation (2). A corresponding installation is also the subject matter of the invention.

### RELATED APPLICATIONS

The present application is a national stage application under 35 U.S.C. § 371 of International Application No. PCT/EP2020/070191, filed 16 Jul. 2020, which claims priority to German Patent Application No. 10 2019 119 540.3, filed 1 Jul. 2019. The above referenced applications are hereby incorporated by reference in their entirety.

### BACKGROUND

The present invention relates to a method for producing a target compound, in particular propylene, and to a corresponding installation in accordance with the preambles of the independent claims.

### PRIOR ART

The production of propylene (propene) is described in the specialist literature, for example in the article “Propylene” in Ullmann's Encyclopedia of Industrial Chemistry, ed. 2012. Propylene is conventionally produced by steam cracking hydrocarbon feeds and conversion processes in the course of refinery processes. In the latter processes, propylene is not necessarily formed in the desired amount and only as one of several components in a mixture with further compounds. Other processes for producing propylene are also known, but are not satisfactory in all cases, for example in terms of efficiency and yield.

An increasing demand for propylene (“propylene gap”), which requires the provision of corresponding selective methods, is predicted for the future. At the same time, it is necessary to reduce or completely prevent carbon dioxide emissions. As a potential starting compound, on the other hand, large amounts of methane are available, which are currently only fed to a material utilization in a very limited manner and are predominantly burned. In addition, appreciable amounts of ethane are often present in corresponding natural gas fractions.

The object of the present invention is to provide a method for the production of propylene, which is improved in particular in view of these aspects, but also for the production of other organic target compounds, in particular of oxo compounds, such as aldehydes and alcohols with a corresponding carbon backbone.

### DISCLOSURE OF THE INVENTION

Against this background, the present invention proposes a method for producing a target compound, in particular propylene, and a corresponding installation with the respective features of the independent patent claims. Preferred embodiments of the present invention are the subject matter of the dependent claims and of the following description.

In principle, in addition to the aforementioned steam cracking processes, a plurality of different methods exist for converting hydrocarbons and related compounds into one another, some of which will be mentioned below by way of example.

For example, the conversion of paraffins to olefins of identical chain length by oxidative dehydrogenation (ODH, also referred to as ODHE in the case of ethane) is known. Typically, a carboxylic acid of identical chain length, i.e. acetic acid in the ODHE, is also formed as a coupling product in the ODH. However, ethylene can also be produced by the oxidative coupling of methane (OCM).

The production of propylene from propane by dehydrogenation (PDH) is also known and represents a commercially available and established process. The same also applies to the production of propylene from ethylene by olefin metathesis. This process requires 2-butene as an additional reagent.

Lastly, so-called methane-to-olefins or methane-to-propylenes (MTO, MTP) processes exist in which synthesis gas is first produced from methane and the synthesis gas is then reacted to give olefins, such as ethylene and propylene. Corresponding processes can be operated on the basis of methane, but also on the basis of other hydrocarbons or carbon-containing starting materials, such as coal or biomass.

Steam reforming and dry reforming as well as modifications thereof, including a downstream water gas shift for adjusting the ratio of hydrogen to carbon monoxide, are likewise known as individual technologies.

Hydroformylation is another technology which is used in particular for the production of oxo compounds of the type mentioned at the outset. Propylene is typically reacted in the hydroformylation, but higher hydrocarbons, in particular hydrocarbons having six to eleven carbon atoms, can also be used. The reaction of hydrocarbons having four and five carbon atoms is also possible in principle, but is of lower practical impact. Hydrogenation can follow the hydroformylation in which aldehydes can initially be formed. Alcohols formed by such hydrogenation can be subsequently dehydrated to give the respective olefins.

In Green et al., Catal. Lett. 1992, 13, 341, a process for the production of propanal from methane and air is described. In the process presented, low yields based on methane are generally observed. In the process, oxidative coupling of methane (OCM) and partial oxidation of methane (PDX) to hydrogen and carbon monoxide are carried out, which are then followed by hydroformylation. The target product is the aforementioned propanal which has to be isolated as such. A limitation arises from the oxidative coupling of methane to give ethylene, for which, at present, typically only lower conversions and limited selectivities are achieved.

The hydroformylation reaction in the aforementioned process is carried out on a typical catalyst at 115° C. and 1 bar in an organic solvent. The selectivity with respect to the (undesirable) by-product ethane is in the range of approx. 1% to 4%, whereas the selectivity with respect to propanal should achieve more than 95%, typically more than 98%. Extensive integration of process steps or the use of the carbon dioxide formed in large amounts as a by-product, in particular in the oxidative coupling of methane, is not described further here, and so there are thus disadvantages compared with conventional processes. Since partial oxidation is used in the process as a downstream step for oxidative coupling, that is to say there is a sequential interconnection, large amounts of unreacted methane in the partial oxidation have to be managed or separated off in a complex manner from the oxidative coupling.

U.S. Pat. No. 6,049,011 A describes a process for the hydroformylation of ethylene. The ethylene can in particular be formed from ethane. Besides propanal, propionic acid can also be produced as the target product. Dehydration is also possible. However, this document does not disclose any further integration and does not disclose any meaningful utilization of the carbon dioxide formed.

### Advantages of the Invention

Against this background, the present invention proposes a method for producing a target compound, in particular propylene, wherein a paraffin, in particular a linear paraffin, more particularly ethane, is subjected to an oxidative dehydrogenation with oxygen to obtain an olefin, in particular a linear olefin, more particularly ethylene.

As already mentioned at the outset, the oxidative dehydrogenation is a process which is, in principle, known from the prior art. In the context of the present invention, known process concepts can be used for the oxidative dehydrogenation. For example, in the oxidative dehydrogenation within the scope of the present invention, a method can be used as is disclosed in Cavani et al., Catal. Today 2007, 127, 113. In particular, catalysts containing V, Sr, Mo, Ni, Nb, Co, Pt and/or Ce and other metals can be used in conjunction with silicate, aluminum oxide, molecular sieve, membrane, and/or monolith carriers. For example, combinations and/or oxides of corresponding metals, for example MoVTeNb oxides and mixed oxides of Ni with Nb, Cr and V, can also be used in the context of the present invention. Examples are described in Melzer et al., Angew. Chem. 2016, 128, 9019, Gartner et al., ChemCatChem 2013, 5, 3196, and Meiswinkel, “Oxidative Dehydrogenation of Short Chain Paraffines”, DGMK-Tagungsbericht 2017-2, ISBN 978-3-941721-74-6, and various patents and patent applications of the applicant.

In addition to the specific composition of the catalysts, particularly in the case of the mentioned MoVTeNb catalysts, the specific crystal arrangement also represents a key feature for achieving high selectivities at high conversions. Among the known catalysts, the mixed oxide catalysts mentioned have a high selectivity and activity in the oxidative dehydrogenation of ethane to give ethylene. It is generally accepted that the crystal phase M1 is responsible for the outstanding catalytic power and selectivity, since it represents the only phase which is capable of abstracting the hydrogen from the paraffin, which represents the first reaction step.

A typical by-product of the oxidative dehydrogenation in essentially all process variants is the respective carboxylic acid, i.e. acetic acid in the case of the oxidative dehydrogenation of ethane, which optionally has to be separated off, but optionally represents a further valuable product and is typically present in contents of a few percent (up to the low two-digit percentage range). Carbon monoxide and carbon dioxide are also formed in the low percentage range. A typical product mixture of the oxidative dehydrogenation of ethane has, for example, the following mixture proportions (preferred value ranges are given in parentheses):

Ethylene 25 to 75 mole percent (30 to 60 mole percent)
Ethane 25 to 70 mole percent (30 to 50 mole percent)
Acetic acid 1 to 20 mole percent (5 to 15 mole percent)
Carbon monoxide 0.5 to 10 mole percent (1 to 5 mole percent)
Carbon dioxide 0.5 to 10 mole percent (1 to 5 mole percent)

These and the following data relate to the dry portion of the product mixture, which, depending on the process regime, can also additionally comprise steam. Further components such as oxygenates, i.e. aldehydes, ketones, ethers, etc., may be present in traces, i.e. typically less than 0.5 mole percent, in particular less than 0.1 mole percent in total.

In the context of the present invention, the olefin formed in the oxidative dehydrogenation is subjected to hydroformylation with carbon monoxide and hydrogen to obtain an aldehyde.

Processes for hydroformylation are also known in principle from the prior art. In recent times, in corresponding processes, as described in the literature cited below, Rh-based catalysts are typically being used. Older methods also employ Co-based catalysts.

For example, homogeneous, Rh(I)-based catalysts with phosphine and/or phosphite ligands can be used. These may be monodentate or bidentate complexes. For the production of propanal, reaction temperatures of 80 to 150° C. and corresponding catalysts are typically used. All methods known from the prior art can also be used in the context of the present invention.

The hydroformylation typically operates at a hydrogen to carbon monoxide ratio of 1:1. However, this ratio can be, in principle, in the range from 0.5:1 to 10:1. The Rh-based catalysts used may have a Rh content of from 0.01 to 1.00% by weight, wherein the ligands may be present in excess. Further details are described in the article “Propanal” in Ullmann's Encyclopedia of Industrial Chemistry, ed. 2012. The invention is not limited by the cited process conditions.

In a further process, as described, for example, in the chapter “Hydroformylation” in Moulijn, Makee & van Diepen, Chemical Process Technology, 2012, 235, a pressure of 20 to 50 bar is used with a Rh-based catalyst and a pressure of 70 to 200 bar is used with a Co-based catalyst. Co also appears to be relevant in metallic form for hydroformylation. Other metals are more or less insignificant, especially Ru, Mn and Fe. The temperature range used in said method is between 370 K and 440 K.

In the process disclosed in the chapter “Synthesis involving Carbon Monoxide” in Weissermel & Arpe, Industrial Organic Chemistry 2003, 135, mainly Co- and Rh-phosphine complexes are used. With specific ligands, hydroformylation can be carried out in aqueous medium and recovery of the catalyst is readily possible.

According to Navid et al., Appl. Catal. A 2014, 469, 357, in principle all transition metals capable of forming carbonyls can be used as potential hydroformulation catalysts, wherein an activity as per Rh>Co>Ir, Ru>Os>Pt>Pd>Fe>Ni is observed according to this publication.

By-products in the hydroformylation are formed in particular by the hydrogenation of the olefin to give the corresponding paraffin, i.e., for example, from ethylene to ethane, or the hydrogenation of the aldehyde to give the alcohol, i.e. from propanal to propanol. According to the article “Propanols” in Ullmann's Encyclopedia of Industrial Chemistry, ed. 2012, propanal formed by hydroformylation can be used as the main source of 1-propanol in industry. In a second step, propanal can be hydrogenated to give 1-propanol.

In the context of the present invention, the paraffin and olefin have a carbon chain having a first carbon number, and the aldehyde has a carbon chain having a second carbon number which is one greater than the first carbon number due to the chain extension in the hydroformylation. The present invention is described below predominantly with reference to ethane as paraffin and ethylene as olefin, but can in principle also be used with higher hydrocarbons.

In the oxidative dehydrogenation, carbon dioxide is formed as a by-product, as mentioned, and the by-product carbon dioxide, which is contained in the aforementioned contents in a corresponding product mixture, is subjected according to the invention at least in part with methane to a dry reforming to obtain carbon monoxide. Since the content of carbon dioxide in a corresponding product mixture is typically in the single-digit percentage range, further carbon dioxide from other sources in addition to the carbon dioxide from the oxidative dehydrogenation can be supplied to the dry reforming at any time. However, the invention always includes the carbon dioxide formed as a by-product of the oxidative dehydrogenation being at least partially supplied to the dry reforming.

Dry reforming is also a method known in principle from the prior art. Reference is made by way of example to Halmann, “Carbon Dioxide Reforming. Chemical fixation of carbon dioxide: methods for recycling CO₂ into useful products”, CRC Press 1993, ISBN 978-0-8493-4428-2, instead of to many. Dry reforming is also referred to as carbon dioxide reforming. In dry reforming, carbon dioxide is reacted with hydrocarbons, such as methane. Here, hydrogen and carbon monoxide and also unreacted carbon dioxide and optionally synthesis gas containing hydrocarbons used are formed, as is conventionally produced by steam reforming. In dry reforming, the reagent steam is replaced to some extent by carbon dioxide. In dry reforming, one molecule of carbon dioxide is reacted with one molecule of methane to give two molecules of hydrogen and two molecules of carbon monoxide. A certain challenge in dry reforming is the comparatively simple further reaction of the formed hydrogen with carbon dioxide to give water and carbon monoxide.

Pressures of up to 40 bar and temperatures of up to 950° C. are typically used in dry reforming. Dry reforming is typically carried out using Ni or Co catalysts or bimetallic catalysts having Ni and Co. Further details are described, for example, in the articles “Gas Production: 2. Processes” and “Hydrogen: 2. Production” in Ullmann's Encyclopedia of Industrial Chemistry, ed. 2012, and in the chapter “Synthesis Gas” in Weissermel & Arpe, Industrial Organic Chemistry, 2003, 15. Embodiments, in particular with respect to the catalysts mentioned, can also be found, for example, in San-José-Alonso et al., Appl. Catal. A, 2009, 371, 54, and Schwab et al., Chem. Ing. Tech. 2015, 87, 347.

As mentioned, in embodiments of the present invention, a hydrogenation and dehydration of the components formed in the hydroformylation can also occur for the production of further products.

Hydrogenation of different unsaturated components is a well known and established technology for converting components having a double bond into the corresponding saturated compounds. Typically, very high or complete conversions with selectivities of well above 90% can be achieved. Typical catalysts for the hydrogenation of carbonyl compounds are based on Ni, as is also described, for example, in the article “Hydrogenation and Dehydrogenation” in Ullmann's Encyclopedia of Industrial Chemistry, ed. 2012. Noble metal catalysts can also be used specifically for olefinic components. Hydrogenations are part of the standard reactions of technical chemistry, as also shown, for example, in M. Baerns et al., “Beispiel 11.6.1: Hydrierung von Doppelbindungen” [“Example 11.6.1: hydrogenation of double bonds”], Technische Chemie 2006, 439. In addition to unsaturated compounds (understood here are olefins, in particular), the authors also mention other groups of substances, such as, for example, aldehydes and ketones in particular as substrates for hydrogenation. Low-boiling substances such as butyraldehyde from the hydroformylation are hydrogenated in the gas phase. Here, Ni and certain noble metals, such as Pt and Pd, typically in supported form, are used as hydrogenation catalysts.

For example, in the article “Propanols” in Ullmann's Encyclopedia of Industrial Chemistry, ed. 2012, a heterogeneous gas phase process is described which is carried out at 110 to 150° C. and a pressure of 0.14 to 1.0 MPa at a hydrogen to propanal ratio of 20:1. Reduction takes place with excess hydrogen and the heat of the reaction is dissipated by circulating the gas phases through external heat exchangers or by cooling the reactor in the interior. The efficiency with respect to hydrogen is more than 90%, the conversion of the aldehyde is effected up to 99.9% and alcohol yields of more than 99% result. Widely used commercial catalysts include combinations of Cu, Zn, Ni and Cr supported on aluminum oxide or kieselguhr. Dipropyl ether, ethane and propyl propionate are mentioned as typical by-products which can form in traces. According to the general prior art, the hydrogenation is preferably effected in particular only with stoichiometric amounts of hydrogen or only a low hydrogen excess.

Details of corresponding liquid-phase processes are also given in the literature. These are carried out, for example, at a temperature of 95 to 120° C. and a pressure of 3.5 MPa. Typically, Ni, Cu, Raney nickel or supported Ni catalysts reinforced with Mo, Mn and Na are preferred as catalysts. 1-propanol can be prepared with 99.9% purity, for example. The main problem with the purification of 1-propanol is the removal of water from the product. If, as in one embodiment of the present invention, propanol is dehydrated to give propylene, water is also one of the reaction products in this step, so that water does not have to be removed beforehand. The separation of propylene and water is thus made simple.

Dehydration of alcohols on suitable catalysts to prepare the corresponding olefins is also known. In particular, the production of ethylene (from ethanol) is common and is gaining importance in connection with the increasing production quantities of (bio)ethanol. Commercial use has been achieved by different companies. For example, reference is made to the aforementioned article “Propanols” in Ullmann's Encyclopedia of Industrial Chemistry and Intratec Solutions' “Ethylene Production via Ethanol Dehydration”, Chemical Engineering 120, 2013, 29. Accordingly, the dehydration of 1- or 2-propanol to give propene has no practical value until now. Nevertheless, the dehydration of 2-propanol in the presence of mineral acid catalysts at room temperature or above is very easy to carry out. The reaction itself is endothermic and equilibrium limited. High conversions are favored by low pressures and high temperatures. Typically, heterogeneous catalysts based on Al₂O₃ or SiO₂ are used. In general, several types of acid catalysts are suitable and, for example, molecular sieves and zeolites can also be used. Typical temperatures range from 200 to 250° C. for the dehydration of ethanol or 300 to 400° C. for the dehydration of 2-propanol or butanol. Owing to the equilibrium limiting, the product stream is typically separated off (separation of the olefin product and also at least partially of the water, for example by distillation) and the stream containing unconverted alcohol is recycled to the reactor inlet. In this way, overall very high selectivities and yields can be achieved.

The present invention proposes overall the coupling of oxidative dehydrogenation, a downstream hydroformylation process, and dry reforming. In the context of the present invention, particular advantages result in particular from the fact that dry reforming can be carried out with the carbon dioxide as starting material, which is inevitably formed as a by-product in the oxidative dehydrogenation, and that the remaining components from a product mixture of the oxidative dehydrogenation and components from a product mixture of the dry reforming, the latter optionally after carrying out a water gas shift, can be used in the hydroformylation without complicated cryogenic separation steps. In particular, unreacted paraffins can be carried along in the hydroformylation and subsequent thereto can be separated more easily, or hydrogen formed in the dry reforming can be used for later hydrogenation steps. The unreacted paraffins can be recycled in a simple manner and used again in the reaction feed.

The present invention thus proposes that the carbon dioxide, which is formed as a by-product in the oxidative dehydrogenation, is subjected at least in part with methane to dry reforming to obtain carbon monoxide. In dry reforming, carbon monoxide and/or hydrogen are obtained, preferably both, and the carbon monoxide obtained in dry reforming and/or the hydrogen obtained in dry reforming are in turn at least partially fed to the hydroformylation. The carbon dioxide can be separated off upstream and/or downstream of the hydroformylation. In this way, in the scope of the present invention, a particularly advantageous and value-creating utilization results of the carbon dioxide, which is formed in the oxidative dehydrogenation and which is unavoidable as by-product. The advantages of the invention thus consist in an advantageous use of a (by-)product of one method in the other and an advantageous use of the products of both methods in a downstream step. As mentioned, the wording according to which “the carbon dioxide which is formed as a by-product in the oxidative dehydrogenation is subjected at least in part with methane to dry reforming to obtain carbon monoxide”, does not preclude that further carbon dioxide provided from any desired source can be supplied to dry reforming. This is the case in one embodiment of the present invention.

In a further embodiment of the present invention, dry reforming can be carried out in an electrically heated reactor. As a particular advantage, this results in the avoidance of carbon dioxide emissions from the firing, as a result of which ideally carbon dioxide emissions from the overall process are completely avoided.

As mentioned, in particular a carboxylic acid can be formed as a further by-product in the oxidative dehydrogenation, in particular acetic acid can be formed in the case of ethane as feed in the oxidative dehydrogenation. This acetic acid, together with reaction water, can be separated off comparatively easily from a corresponding product mixture of the oxidative dehydrogenation by condensation and/or a water scrubbing. Owing to its strong interaction with suitable solvents or washing liquids, carbon dioxide can likewise be removed comparatively easily from the product mixture, wherein it is possible to use known methods for removing carbon dioxide, in particular corresponding scrubbing (for example amine scrubbing). Cryogenic separation is not required, so that the entire method of the present invention, at least including dry reforming and hydroformylation, forgoes cryogenic separation steps. Should subsequent steps require the absence of, or only a very low residual concentration of, carbon dioxide (for example due to catalytic inhibition or poisoning), the residual carbon dioxide content after amine scrubbing can be further reduced by an optional caustic scrubbing as fine cleaning, as required.

Any water-containing gas mixtures occurring in the context of the present invention can be subjected to drying at a suitable point in each case. For example, drying can take place downstream of the hydroformylation if, in one embodiment of the present invention, this takes place in the aqueous phase and the hydrogenation downstream of the hydroformylation requires a dry stream as reaction feed. If this is not necessary for the subsequent process steps, drying does not have to take place until complete dryness; rather, water contents can optionally also remain in corresponding gas mixtures, as long as these are tolerable. Different drying steps can also be provided at different points in the method and optionally with different degrees of drying.

The separation of the aforementioned by-products advantageously takes place completely non-cryogenically and is therefore extremely simple in terms of apparatus and in terms of energy expenditure. This represents a substantial advantage of the present invention over prior art methods which typically require complex separation of components that are undesirable in subsequent process steps.

“Non-cryogenic” separation refers to a separation or separation step which is carried out in particular at a temperature level above 0° C., in particular at typical cooling water temperatures of from 5 to 40° C., in particular from 5 to 25° C., optionally also above ambient temperature. In particular, however, non-cryogenic separation in the sense referred to here represents a separation without the use of a C2 and/or C3 cooling circuit and it is therefore carried out above −30° C., in particular above −20° C.

As a further by-product of the oxidative dehydrogenation, unreacted paraffin and carbon monoxide are typically present in a corresponding product mixture. These compounds can be transferred into the subsequent hydroformylation without difficulty. Carbon monoxide can be reacted with the olefin together with carbon monoxide from dry reforming. The paraffin is typically not reacted in the hydroformylation. Since heavier compounds with a higher boiling point or other polarity are formed in the hydroformylation, they can be separated off comparatively easily, and likewise non-cryogenically, from the remaining paraffin.

In the context of the present invention, the aldehyde formed in the hydroformylation can be the target compound or, in the context of the present invention, this aldehyde can be further converted to give an actually desired target compound. The latter variant in particular represents a particularly preferred embodiment of the present invention.

In particular, when the aldehyde is reacted to give the target compound, the aldehyde can first be hydrogenated to give an alcohol which has a carbon chain having the second carbon number, i.e. the same carbon number as the aldehyde. A corresponding process variant is particularly advantageous because it is possible to use for this hydrogen which is contained in a product mixture of the dry reforming and which can be already present in a feed mixture upstream of the hydroformylation and can be passed through the hydroformylation. In the context of the present invention, a content of hydrogen and carbon monoxide in a product mixture of the dry reforming can be adjusted, in particular, in a water gas shift of a type known in principle. The water gas shift can be carried out in particular downstream of the dry reforming and in particular upstream of the hydroformylation. In particular, the water gas shift is carried out before the combining of substance streams from the dry reforming and the oxidative dehydrogenation. By using the water gas shift, the present invention enables a precise adaptation of the respective hydrogen and/or carbon monoxide contents to the respective need in the hydroformylation or the downstream hydrogenation.

Due to a corresponding water gas shift, consideration can also be given in particular to the possible contents of carbon monoxide in a product mixture of the oxidative dehydrogenation, which is combined with carbon monoxide from the dry reforming for use in the hydroformylation. The use of a water gas shift downstream of the dry reforming thus enables an exact adaptation to the respective requirements in the hydroformylation.

Hydrogen can be fed in at any suitable point in the method according to the invention and its embodiments, in particular upstream of the optionally provided hydrogenation. In this way, hydrogen is available for this hydrogenation. The feeding need not take place directly upstream of the hydrogenation; rather, hydrogen can also be fed in by method or separation steps present or carried out upstream of the hydrogenation. Hydrogen can also be separated off, for example, from a partial stream of a product stream from the dry reforming or formed as a corresponding partial stream, for example by separation steps known per se, such as pressure swing adsorption.

In a further embodiment of the present invention, during the reaction of the aldehyde to give the actual target compound of the method according to the invention, a dehydration of the alcohol formed by the hydrogenation to give a further olefin (based on the previous olefin formed in the oxidative dehydrogenation) takes place, wherein the further olefin, in particular propylene, has a carbon chain with the mentioned second carbon number, i.e. the carbon number of the aldehyde formed beforehand and the alcohol formed therefrom.

In particular, the alcohol formed in the reaction of the aldehyde can be separated off comparatively easily from unreacted paraffin. In this way, a recycle stream of the paraffin can also be formed non-cryogenically here and recycled, for example, into the oxidative dehydrogenation.

As already mentioned several times, in the context of the present invention, the first carbon number can be two and the second carbon number can be three; therefore, it is first possible to produce ethylene as olefin from ethane as paraffin in the oxidative dehydrogenation, wherein the ethylene is converted to propanal in the hydroformylation. This propanal can subsequently be reacted by a hydrogenation to give propanol and this in turn can be reacted to give propylene by a dehydration.

In a particularly preferred embodiment, the present invention permits the use of all components of natural gas. For this purpose, raw natural gas can be used and separated into a methane fraction and into a fraction having heavier hydrocarbons, in particular rich in ethane. The methane fraction can be fed to the dry reforming and the fraction with heavier hydrocarbons to the oxidative dehydrogenation. The fraction having heavier hydrocarbons may also be further treated, for example if a substantially pure ethane fraction is to be formed for the oxidative dehydrogenation.

As already mentioned, the carbon monoxide obtained in the dry reforming can be obtained in a product mixture which also contains at least hydrogen. This hydrogen can be passed through the hydroformylation and subsequently used in a hydrogenation. The product mixture from the dry reforming can, as has likewise already been mentioned, be subjected to a water gas shift. In particular, the product mixture from the dry reforming and/or the product mixture from the water gas shift can be subjected, at least partially unseparated, to the hydroformylation.

Further aspects of the present invention have also already been mentioned in principle. In particular, the olefin obtained in the oxidative dehydrogenation may be obtained in a product mixture further containing carbon dioxide and carbon monoxide, wherein the carbon dioxide at a suitable point is at least partially non-cryogenically separated from the product mixture of the oxidative dehydrogenation or a subsequent step and is subjected to the dry reforming. As mentioned, the separation of the carbon dioxide can take place both before and after the hydroformylation. The carbon monoxide and the olefin may be subjected to the hydroformylation at least in part without prior separation from each other. As mentioned, in the context of the present invention, in principle a complete non-cryogenic separation of obtained gas mixtures can be achieved. This is not necessarily the case for the separation of natural gas into the methane fraction and the fraction with heavier hydrocarbons mentioned at the outset.

As already mentioned, at least some of the paraffin may pass through the oxidative dehydrogenation and the hydroformylation unreacted. As mentioned in detail above, this part can be separated off downstream of the hydroformylation and recycled into the oxidative dehydrogenation. The separation can take place directly downstream of the hydroformylation, i.e. before each process step following the hydroformylation, or downstream of a process step following the hydroformylation, for example after hydrogenation or dehydration, but also after any separation or work-up steps.

In a particularly preferred embodiment of the present invention, a product mixture from the oxidative dehydrogenation, in particular after a condensate removal, is compressed to a pressure level at which both the carbon dioxide is separated from the oxidative dehydrogenation before and/or after the hydroformylation and the hydroformylation is carried out. Additional intermediate steps may optionally also be provided between the separation of carbon dioxide and the hydroformylation upstream and/or downstream thereof. Both methods take place essentially at the same pressure level, which means in particular that no additional compression takes place between the two and the precise operating pressure of both steps only results from the process-related pressure losses between the two steps.

The pressure level at which the removal of carbon dioxide and the hydroformylation are operated preferably represents the highest pressure level in the overall process, which means in particular that the dry reforming is carried out at a lower pressure level than the separation of carbon dioxide and the hydroformylation upstream and/or downstream thereof.

In this way, it is possible to dispense with providing otherwise required additional compression steps and corresponding compressors. In the context of the present invention, the oxidative dehydrogenation is advantageously carried out at a pressure level of 1 to 10 bar, in particular 2 to 6 bar, the dry reforming at a pressure level of advantageously 15 to 100 bar, in particular 20 to 50 bar, and the hydroformylation and the removal of carbon dioxide are advantageously carried out at a pressure level of 15 to 100 bar, in particular 20 to 50 bar.

The present invention also extends to an installation for producing a target compound, in relation to which reference is expressly made to the corresponding independent patent claim. A corresponding installation, which is preferably set up for carrying out a method, as has been explained above in different embodiments, benefits in the same way from the advantages already mentioned above.

The invention will be explained in more detail below with reference to the accompanying drawing, which illustrates a preferred embodiment of the present invention.

### BRIEF DESCRIPTION OF THE DRAWINGS

FIG. 1 illustrates a method according to one embodiment of the invention in the form of a schematic flowchart.

If reference is made below to process steps, such as oxidative dehydrogenation, dry reforming or hydroformylation, these are also to be understood to cover the apparatus used in each case for these process steps (in particular, for example, reactors, columns, scrubbing devices, etc.), even if this is not expressly referred to. In general, the explanations relating to the method apply to a corresponding installation in the same way in each case.

### DETAILED DESCRIPTION OF THE DRAWINGS

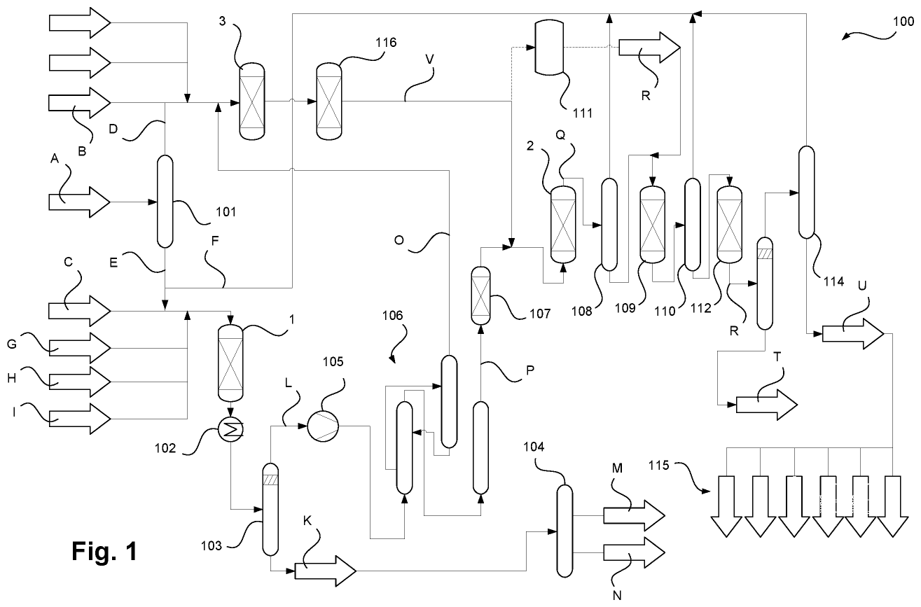

FIG. 1 illustrates a method according to a particularly preferred embodiment of the present invention in the form of a schematic flowchart and is designated overall by 100.

Central process steps or components of the method 100 are an oxidative dehydrogenation, which is designated here overall by 1, and a hydroformylation, which is designated here overall by 2. The method 100 further comprises a dry reforming, designated here overall by 3.

In the example shown, a natural gas stream A is supplied to the method 100. Instead of or in addition to natural gas stream A, however, a separate methane stream B and an ethane stream C can also be provided. The invention is described again here with reference to ethane as paraffin feed, but can, as mentioned, also be used in the case of higher paraffins. Further, in the example illustrated here, a vapor stream B1 and a carbon dioxide stream B2 are provided from an external source.

The natural gas stream is first subjected to fractionation 101, in particular in a corresponding column, a methane stream being obtained as overhead product and a material stream containing the heavier hydrocarbons of the natural gas stream, in particular ethane, being obtained as bottom product. The overhead stream is denoted by D here and the bottom stream by E. The material stream E, which may also predominantly or exclusively contain ethane, is fed together with a recycle stream F to the oxidative dehydrogenation 1. In this case, mixing with oxygen, which is provided in the form of a material stream G, and with vapor, which is provided in the form of a material stream H, is carried out. The vapor of the material stream H, like nitrogen of an optionally provided nitrogen stream I, serves as a diluent or moderator and in this way prevents in particular a thermal runaway in the oxidative dehydrogenation 1. Particularly in the case of the aforementioned MoVTeNb mixed metal oxide catalysts, steam furthermore has the function of ensuring catalyst stability (long-term performance), and a moderation of the catalytic selectivity is possible by means of steam.

Downstream of the oxidative dehydrogenation, an aftercooler 102 is provided downstream of which there is, in turn, a condensate separation 103. A condensate stream K formed in the condensate separation 103, which predominantly or exclusively contains water and acetic acid, can be fed to an acetic acid recovery 104 in which, in particular, a water stream M and an acetic acid stream N are formed.

The product mixture of the oxidative dehydrogenation 1 freed of condensate is compressed in the form of a material stream L in a compressor 105 and subsequently supplied to a carbon dioxide removal, designated overall by 106, which can be carried out, for example, using corresponding scrubbing. In the embodiment shown here, a scrubbing column 106a for an amine scrubbing and the regeneration column 106b for the amine-containing scrubbing liquid loaded with carbon dioxide in the scrubbing column 106a are shown. An optional scrubbing column 106c for fine purification, for example for a caustic scrubbing, is also shown. As mentioned, carbon dioxide removal and recovery by appropriate scrubbing is generally known. It is therefore not explained separately.

A carbon dioxide stream O formed in the carbon dioxide removal 106 can, as explained further below, be fed into the dry reforming 3.

A mixture of components remaining in the form of a material stream P, after the removal of carbon dioxide in the carbon dioxide removal 106, contains predominantly ethylene, ethane and carbon monoxide. It is optionally dried in a dryer 107 and subsequently supplied together with a further material stream V (see below) to the hydroformylation 2.

In the hydroformylation 2, propanal is formed from the olefins and carbon monoxide and hydrogen, which together with the further components explained is carried out in the form of a material stream Q from the hydroformylation 2. Unreacted ethane, in particular ethane unreacted in the oxidative dehydrogenation 1, can optionally be separated from the material stream Q in a separation 108 and this ethane can be transferred into the recycle stream F. This recycle stream F also contains any further substances present and which boil more easily than propanal. Alternatives to the separation 108 are discussed further below, however, the separation 108 is a preferred embodiment.

In a hydrogenation 109, the propanal can be converted to propanol. The alcohol stream is fed to a further separation 110, optionally provided as an alternative to the separation 108, where ethane, in particular ethane unreacted in the oxidative dehydrogenation 1, and any further substances present, can be separated more easily than propanol and transferred into the recycle stream F.

The hydrogenation 109 can be operated with hydrogen which is contained in a product stream of the dry reforming 3 and is carried along in the hydroformylation. Alternatively, the separate feeding of required hydrogen in the form of a material stream R is also possible, in particular from a separation of hydrogen in a pressure swing adsorption 111.

A product stream from the hydrogenation 109 or from the optionally provided separation 110 is fed to a dehydration 112. In said dehydration, propylene is formed from the propanol. A product stream S from the dehydration 112 is fed to a condensate separation 113 where it is freed of condensible compounds, in particular water. The water can be carried out of the process in the form of a water stream T. The water streams N and T can, optionally after a suitable work-up, also be fed again to the process for steam generation. In this way, for example, at least a part of the steam flow B1 can be provided.

The gaseous residue remaining after the condensate separation 113 is fed to a further separation 114 optionally provided as an alternative to the separations 108 and 110 where, in turn, ethane unreacted particularly in the oxidative dehydrogenation 1 can be separated off and transferred into the recycle stream F. A product stream U formed in the separation 114 can be carried out of the process and used in further process steps, for example for the production of plastics or other further compounds, as indicated here overall by 115. Corresponding methods are known per se in a variety of forms and comprise the use of the propylene from the method 100 as intermediate product or starting product in the petrochemical value chain.

Ethane unreacted in the oxidative dyhdrogenation 1 is, as mentioned several times, recycled with the material stream F into the oxidative dehydrogenation 1.

A water gas shift 116 is optionally connected downstream of the dry reforming 3. A product mixture V formed in each case in the dry reforming 3 or the (optional) water gas shift 116, which predominantly or exclusively contains hydrogen and carbon monoxide, is fed (after an optional hydrogen separation in the pressure swing adsorption 111), together with the material stream P freed of carbon dioxide, from the oxidative dehydrogenation 1 to the hydroformylation 3.

1. A method for producing a target compound, the method comprising:
subjecting a paraffin to an oxidative dehydrogenation process with oxygen to obtain an olefin,
subjecting the olefin to a hydroformylation process with carbon monoxide and hydrogen to obtain an aldehyde, wherein the paraffin and the olefin comprise a first carbon chain having a first carbon number and wherein the aldehyde comprises a second carbon chain having a second carbon number, wherein the second carbon number is one greater than the first carbon number,
forming in the oxidative dehydrogenation process carbon dioxide as a by-product, subjecting the carbon dioxide at least in part to a dry reforming process with methane to obtain carbon monoxide and hydrogen, and
supplying at least in part to the hydroformylation process, at least one selected from the group of the carbon monoxide or hydrogen obtained in the dry reforming process or mixtures thereof.

2. The method of claim 1, wherein the aldehyde is the target compound, or further comprising reacting the aldehyde to produce the target compound.

3. The method of claim 2 further comprising, reacting the aldehyde in a hydrogenation process to produce an alcohol having a third carbon chain having the second carbon number.

4. The method of claim 3 further comprising:
reacting the alcohol in a dehydration process to produce a second olefin having a fourth carbon chain having the second carbon number.

5. The method according to claim 1, wherein the first carbon number is two and the second carbon number is three.

6. The method of claim 1 further comprising:
separating the methane and the paraffin from natural gas.

7. The method of claim 1 wherein the carbon monoxide obtained in the dry reforming process is obtained in a first product mixture comprising hydrogen.

8. The method of claim 7 further comprising:
subjecting the first product mixture from the dry reforming process to a water gas shift to obtain a second product mixture.

9. The method of claim 8 further comprising:
supplying a third product mixture comprising at least one selected from the group of the first product mixture from the dry reforming process and the second product mixture from the water gas shift to the hydroformylation process, wherein the first product mixture and the second product mixture are at least partially unseparated.

10. The method of claim 1 further comprising:
obtaining the olefin obtained in the oxidative dehydrogenation process in a product mixture, wherein the product mixture comprises the olefin, carbon dioxide, and carbon monoxide,
separating off at least partially the carbon dioxide at a location chosen from upstream, or downstream, or combinations thereof relative to the hydroformylation process,
subjecting the carbon dioxide to the dry reforming process, and
subjecting at least partially the carbon monoxide and the olefin to the hydroformylation process without prior separation from each other.

11. The method according to claim 1 further comprising:
passing at least part of the paraffin through the oxidative dehydrogenation process and the hydroformylation process unreacted,
separating the paraffin off downstream of the hydroformylation process, and
recycling the paraffin into the oxidative dehydrogenation process.

12. The method of claim 1 further comprising:
compressing the product mixture from the oxidative dehydrogenation to a pressure level at which the carbon dioxide is separated off and the hydroformylation process is carried out, and wherein the dry reforming process is carried out at a lower pressure level.

13. The method of claim 1, wherein the method is carried out completely non-cryogenically downstream of the oxidative dehydrogenation process and the dry reforming process.

14. A system for producing a target compound comprising:
the system configured to subject a paraffin to an oxidative dehydrogenation process with oxygen to obtain an olefin, and to subject the olefin to a hydroformylation process with carbon monoxide and hydrogen to obtain an aldehyde, wherein the paraffin and the olefin have a carbon chain having a first carbon number and the aldehyde has a second carbon chain having a second carbon number, wherein the second carbon number is one greater than the first carbon number,
the system further configured to form carbon dioxide as a by-product in the oxidative dehydrogenation process, and further configured to subject the carbon dioxide to a dry reforming process at least in part with methane to obtain carbon monoxide and hydrogen, and to supply at least one of the carbon monoxide or hydrogen obtained in the dry reforming process, or mixtures thereof at least in part to the hydroformylation process.

15. (canceled)
